(19) United States
(12) Patent Application Publication
(10) Pub. No.: US 2017/0191131 A1
(43) Pub. Date: Jul. 6, 2017

(54) PROBES TO FIBRONECTIN AND FIBROBLAST GROWTH FACTOR RECEPTOR GENES

(71) Applicant: CYTOTEST INC., Rockville, MD (US)

(72) Inventors: Reinhard Ebner, Gaithersburg, MD (US); Hua ZOU, Rockville, MD (US); Chi-Chung Cheng, Rockville, MD (US)

(73) Assignee: Cytotest, Inc., Rockville, MD (US)

(21) Appl. No.: 15/398,050

(22) Filed: Jan. 4, 2017

Related U.S. Application Data
(60) Provisional application No. 62/274,473, filed on Jan. 4, 2016.

(51) Int. Cl. C12Q 1/68; A61K 31/506

(52) U.S. Cl. CPC C12Q 1/6883; A61K 31/506; C12Q 2600/156

(57) ABSTRACT

A detectably labeled DNA FISH probe comprises detectably-labeled nucleic acid molecules from the 8p11.23 chromosomal region and from the 2q35 chromosomal region. The probe is a FISH probe capable of detecting a fusion between the FGFR1 and FN1 genes. A patient having a disease associated with a FN1-FGFR1 gene fusion may be treated by: detecting translocation of a portion of the FGFR1 gene to the FN1 gene using chromosomal FISH probes, and treating a patient with said translocation and gene fusion with 1) a drug which inhibits FGF 2) a drug which inhibits FGFR1, and/or a 3) drug which targets a signaling pathway associated with FGFR1.

### CROSS-REFERENCE TO RELATED APPLICATIONS

This application claims the benefit of U.S. Provisional Application No. 62/274,473, filed Jan. 4, 2016, entitled “PROBES TO FIBRONECTIN AND FIBROBLAST GROWTH FACTOR RECEPTOR GENES,” which is hereby incorporated by reference in its entirety.

### BRIEF DESCRIPTION OF THE SEVERAL VIEWS OF THE DRAWINGS

FIG. 1 illustrates the design of a detectably labeled FISH probe for detecting a fusion between the FN1 gene and FGFR1 gene.

FIG. 2 is a table of showing a list of BAC clones to the 8p11.23 region (FGFR1) flanking the 3′ portion of the chromosomal break.

FIG. 3 is a table showing a list of BAC clones to the 8p11.23 region (FGFR1) flanking the 5′ portion of the break.

FIG. 4 is a table showing a list of BAC clones to the 2q35 region (FN1).

### DETAILED DESCRIPTION OF EMBODIMENTS

The present invention relates to probes directed to portions of the human fibronectin and fibroblast growth factor receptor genes. The probes are particularly useful as a companion diagnostic to accompany the treatment and/or diagnosis of diseases associated with genetic defects in the fibronectin and/or fibroblast growth factor receptor genes, particularly a defect in which a portion of the human FN1 (fibronectin) gene is fused to a portion of the human FGFR1 (fibroblast growth receptor) gene. Such diseases and conditions include, without limitation, cancers, diseases and conditions associated with activation of fibroblast growth factor 1, diseases and conditions associated with high levels of fibroblast growth factor 23 (“FGF23”), cancers, diseases and conditions associated with a genetic rearrangement of the FN1 and/or FGFR1, such as phosphaturic mesenchymal tumor, and any diseases and conditions associated with aberrant vitamin D and phosphate metabolism, bone conditions, etc.

A companion diagnostic is particularly useful because it facilitates personalized and targeted drug therapy in which a patient is treated with the drug most likely to be effective to treat and/or prevent the disease. It is based on the knowledge of the genetic or physiological defect underlying the disease, permitting a clinician to make an informed decision about the therapeutic course.

The present invention relates to nucleic acid probes for detecting nucleic acid sequences. The sequences can be present in any sample of interest, including in tissues, cells, organelles, chromosomes, biopsy samples, tissues present on slides, skin, hair, environmental, soil, clothing, forensic samples, blood, saliva, tissue exudate, etc.

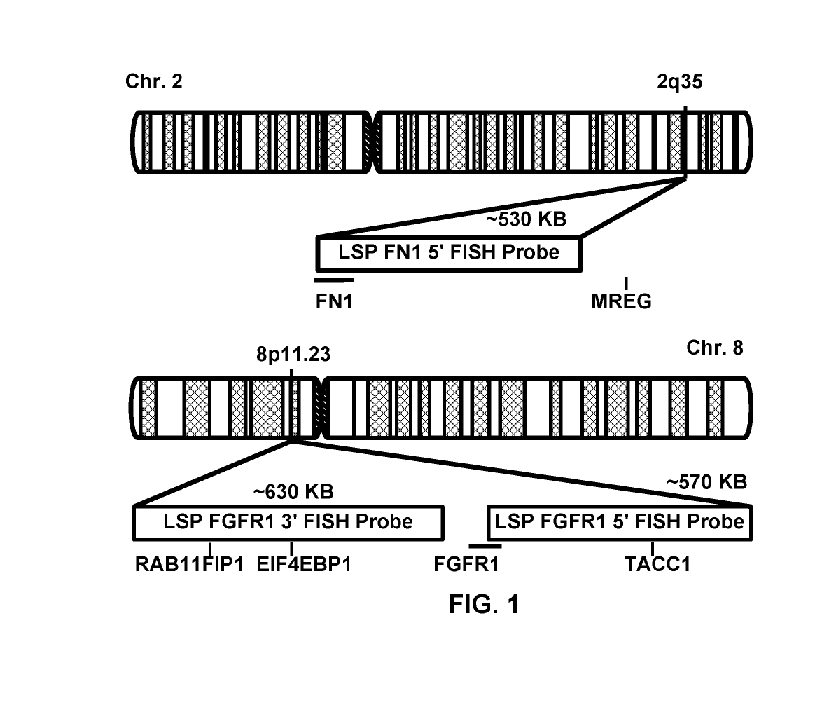

FIG. 1 shows the design of a detectably labeled FISH probe for detecting a fusion between the FN1 gene and FGFR1 gene. The probe comprises DNA from FN1 5′ region and the 3′ and 5′ regions of the FGFR1 gene as shown in the figure.

Generally, a target acid nucleic acid is selected for detection. For example, it may be desired to determine whether a chromosomal fragment (e.g., containing one or more genes) has been amplified, rearranged or deleted in a chromosome, translocated to another chromosome, mutated, etc. In the case of amplification, the copy number of one or more genes in the fragment can be increased in comparison to other genes present on the chromosome. Amplification generally involves multiplication of a region of the chromosome resulting in an increase in the copy number of the genes in the amplified region. Genes can also become rearranged, including by deletions, insertions, fusions, translocations, and other aberrations, e.g., involving other genes and chromosomal regions.

The probes of the present invention are useful for detecting any of the above-mentioned changes in a genome, including chromosomes, mitochondrial DNA, extra-nuclear genomes, bacterial chromosomes, etc. In addition, the probes are useful in detecting aneuploidy, such as trisomy, where karyotyping is typically utilized to detect genetic abnormalities.

To make a nucleic acid probe in accordance with the present invention, a specific region is selected as a target to be detected. The target can be of any desired size, e.g., the entire region, a part of a region, or a specific gene or genes. Once the target is identified, the nucleic acid from the region is obtained for preparation of the nucleic acid probe.

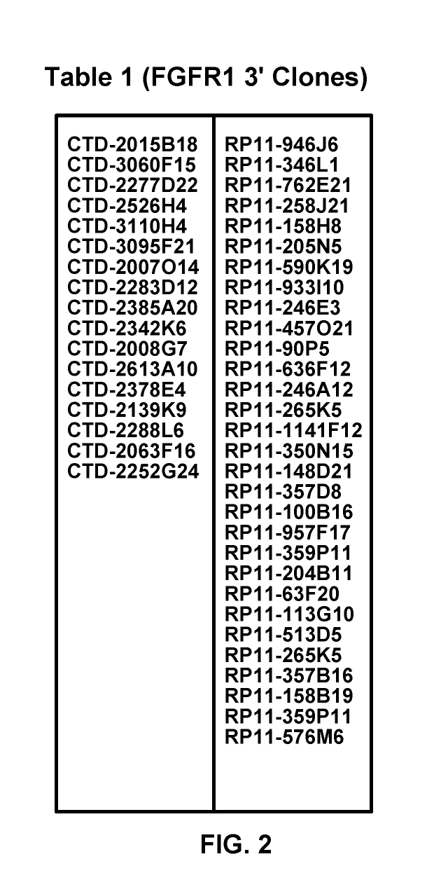

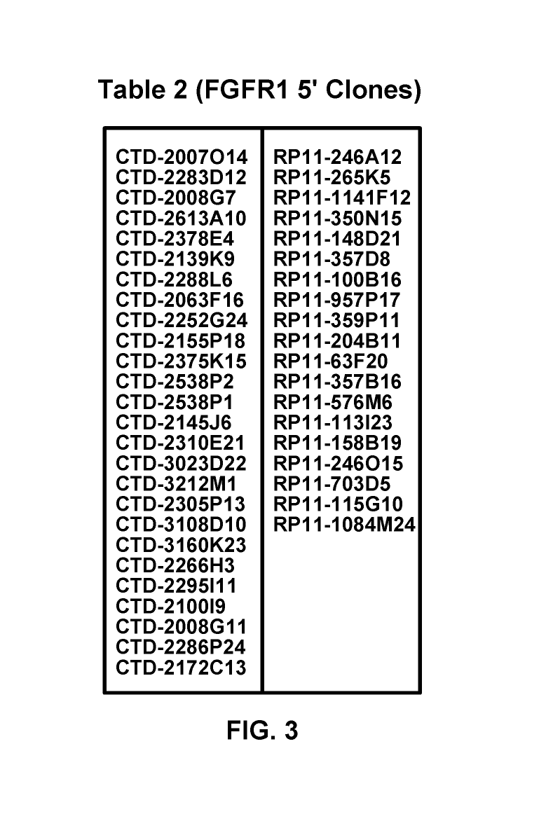

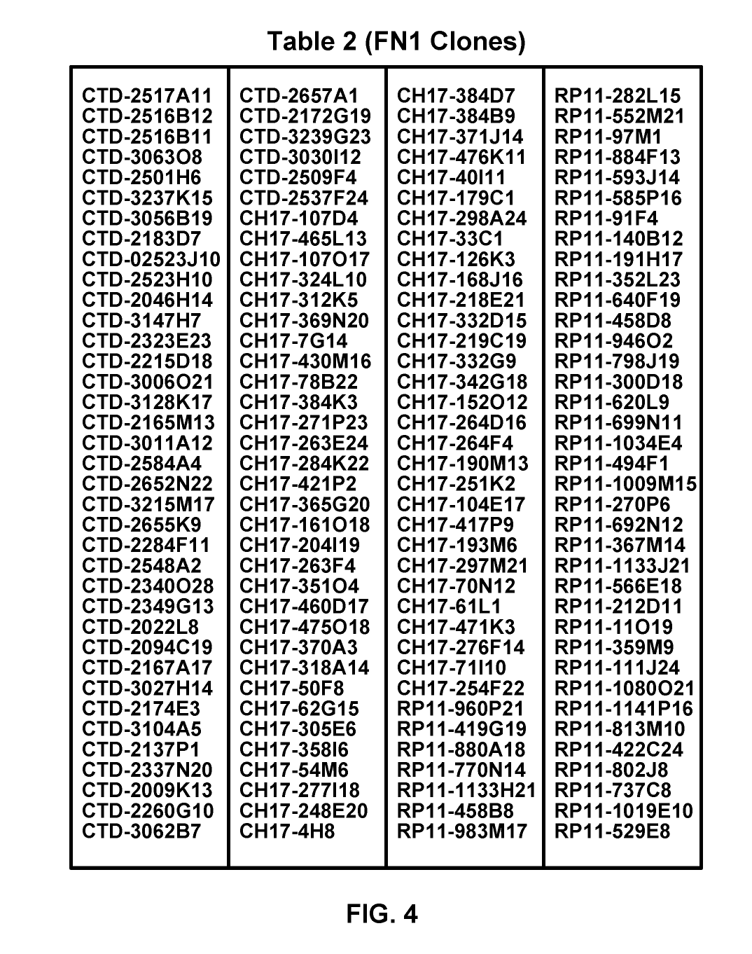

There are a variety of difference sources from which the nucleic acid can be obtained. These include, but are not limited to, BAC (bacterial artificial chromosome) libraries, YAC (yeast artificial chromosome) libraries, PCR (polymerase chain reaction) product fragments, bacteriophage libraries, plasmid libraries, cDNA libraries, genomic libraries, libraries made from dissected chromosomal regions. Tables 1 (FIG. 2), Table 2 (FIG. 3) and Table 3 (FIG. 4) in FI provide examples of BAC clones that can be used to manufacture probes in accordance with the present invention.

The probe can be prepared by any suitable method. Generally, once the nucleic acid to be used as a probe source is obtained, it will be amplified to increase its amount, e.g., by PCR, nick-translation, random priming, etc. Probes can be prepared as described in, e.g., Rigby et al. J. Mol. Biol., 113:237 (1977); Sambrook et al., Molecular Cloning—A Laboratory Manual, Cold Spring Harbor Laboratory, Cold Spring Harbor, N.Y. (1985); US20140256575A1

Probes prepared in accordance with the above-mentioned methods can incorporate naturally-occurring and non-naturally occurring nucleotides. Examples of non-naturally occurring nucleotides useful in the present include, but are not limited to, nucleotides which are disclosed in U.S. Pat. Nos. 5,476,928, 5,449,767, 5,328,824, and 8,058,031. Non-naturally occurring nucleotides include nucleotides comprising a detectable label, such as fluorophore or other reporter label. Particularly useful are labeled non-naturally occurring nucleotides which are incorporated by a polymerase into the nucleic acid strand. See Anderson et al., BioTechniques, 38(2):257-263 (2005).

Preferably, the probes are labeled with detectable labels to enable detection of the probe. The probe can be labeled prior to its hybridization with a target, during hybridization, or after hybridization. Detectable labels and methods of labeling nucleic probes are well known in the art. Detectable labels can be incorporated during the probe manufacture process, e.g., incorporating modified nucleotides which comprise a detectable label or a linker molecule (such as avidin or biotin) that enable a label to be attached to the nucleotide once the probe is made. Labeling protocols are well-known in the art, e.g., as described in Smith, Methods Mol. Biol. 18:445-447 (1993).

Useful detectable labels include, but are not limited to: fluorescent dyes, biotin, enzymes, fluorescein, Texas Red, DNP. Useful labeled nucleotides include, biotin-16-dUTP, 7-amino-4-methylcoumarin-3-acetic acid (AMCA)-6-dUTP, fluorescein-11-dUTP, tetramethyl-rhodamine (TMR)-6-dUTP, digoxigenin-11-dUTP, fluorescein-dATP, and IR-770-dATP.

The nucleic acid probes of the present invention are preferably non-naturally occurring. Specifically, in preferred embodiments, the probes are not directed to contiguous and connected chromosomal regions, but rather are fragmented portions of the desired region. For example, for chromosomal region 2q34 or 8p11 to detect FN1-FGFR1 fusion, the probe does not comprise molecules which are continuous or contiguous with a genomic sequence from that region, but rather contain non-continuous fragments from it, such that the probe is made of up a regions of the 2q34 or 8p11 where the entire locus is not completely represented in the probe, but is missing certain portions of it.

The probe also preferably does not contain equal representations or proportions of each sub-region within the target region and is therefore not naturally-occurring or a representation of a naturally-occurring gene or gene sequence. For example, if chromosome band 2q34 comprising the FN1 gene is selected, the probe can contain fragments of the region in unequal quantities, i.e., if the region is divided into ten different fragments, fragment 1 may be present in 1× quantity, fragment 2 in 2× quantity, fragment 3 in 1× quantity, fragment 4 in 4× quantity, and so on. Such unequal representations of the chromosomal region as it occurs in nature can be produced by several different methods. For example, if overlapping BAC clones are utilized to prepare a probe from a desired genomic region, the regions of overlap will be present in greater amounts than non-overlapping regions and therefore will be amplified unequally and to a greater extent than the non-overlapping regions. The same result can be obtained by starting with certain BAC clones, etc. present in larger amounts than other BAC clones in the same target region. Regions can also be prepared individually, and then pooled in differing amounts (region 1 in quantity x; region 2 in quantity 2×, etc.). Significantly, probes prepared in this manner are not products of nature and are compositions in a form that would not be found in nature.

Hybridization

Once a probe is produced as described above, it can be used to detect the target nucleic acid in a sample. Generally, the probe is used as a hybridization probe in any suitable format. Formats include, without limitation, liquid hybridization, PCR, Southern, Northern, microarrays, microscope slides, paraffin sections, cryosections.

Hybridization conditions are well known in the art. See, e.g., Wangsa et al., Am. J. Pathol., 175(6): 2637-2645, Dec. 2009. Hybridizations conditions can be varied by changing the composition of the hybridization solution, e.g., by varying pH, salt concentration, detergent concentration, buffer concentration and type (e.g., phosphate, Tris(hydroxymethyl)aminomethan), chelation concentration (e.g., EDTA), formamide, dextran sulphate, DTT, Denhardt's solution, etc.

As indicated above, the probe can be pre-labeled, such that after hybridization is complete and unbound probe is washed away, the probe can be immediately detected. In another embodiment, detectable label can be added to the probe after it is bound to the target nucleic acid.

An extensive guide to the hybridization of nucleic acids is found in, e.g., Tijssen (1993) Laboratory Techniques in Biochemistry and Molecular Biology—Hybridization with Nucleic Acid Probes part I, Ch. 2, “Overview of principles of hybridization and the strategy of nucleic acid probe assays,” Elsevier, NY.

In Situ Hybridization

In situ hybridization (ISH) is a technique that involves hybridizing a probe to a target nucleic acid in which the target is present in a tissue preparation, tissue section (paraffin, plastic, cryo, etc.), cells, embryos, mitotic chromosomal spread, etc. In this technique, the target is detected in situ in the location where it is normally found. For example, the target can be detected in the cell cytoplasm, in an organelle (e.g., mitochondria or chloroplast), or in the chromosomal DNA. The chromosomal DNA in general is an intact chromosome that can be present in the tissue section or cell in its intact form or it can be isolated. In each case, the sample containing the target nucleic acid is treated in such a way that the probe can access the target chromosome or chromosome fragment, hybridize to it, and then be detected. When the probe is fluorescently labeled, the technique is known as fluorescence in situ hybridization (FISH).

ISH can be carried out conventionally, e.g., as described in In Situ Hybridization Protocols, Edited by B. Nielsen, Humana Press (Fourth Edition) (2014).

ISH can be performed with one or more detectable labels. For example, M-FISH (multiplex fluorescence or multi-color or multispectral FISH) is a technique in which multiple probes, each of which binds to a different DNA sequence and each of which bears a different detectable label, is used to detect multiple different sequences on the same sample, for example, on the same chromosome. M-FISH is useful for looking a chromosome rearrangements or translocations, or looking at independent loci in the same sample. See, e.g., U.S. Pat. No. 5,880,473 for the use of multiple filters in M-FISH. For SKY (spectral karyotyping), in which each chromosome pair is visualized in a different color, see, e.g., Schröck E, du Manoir S, Veldman T, Schoell B, Wienberg J, Ferguson-Smith M A, Ning Y, Ledbetter DH, Bar-Am I, Soenksen D, Garini Y, Ried T. Multicolor spectral karyotyping of human chromosomes. Science 273:494-497, 1996.

Generally, a dye exhibiting a specific wavelength is chosen as a label. A given dye is characterized by an excitation (absorption) spectrum and an emission spectrum. The excitation and emission spectra are also sometimes referred to as the excitation and emission bands. When the dye is irradiated with light at a wavelength within the excitation band, the dye fluoresces, emitting light at wavelengths in the emission band. Thus, when the sample is irradiated with excitation radiation in a frequency band that excites a given dye, portions of the sample to which the probe labeled with the given dye is attached will fluoresce. If the light emanating from the sample is filtered to reject light outside the given dye's emission band, and then imaged, the image nominally shows only those portions of the sample that bind the probe labeled with the given dye.

The term “hybridization” refers to the specific binding of a nucleic acid to a complementary nucleic acid via Watson-Crick base pairing. The term “in situ hybridization” refers to specific binding of a nucleic acid to a target nucleic acid in its normal place in a sample, such as on metaphase or interphase chromosomes. The terms “hybridizing” and “binding” are used interchangeably to mean specific binding between a nucleic acid probe and its complementary sequence.

The term “chromosomal region” means a contiguous length of nucleotides in the genome of an organism. A chromosomal region may be in the range of 10 kb in length to less than a complete chromosome, e. g., 100 kb to 10 MB for example. FISH probes are most typically in the 50 kpb to 1000 kbp length range, but can be lower as well, e.g., 10, 15, 20, 25, 30, 40, 45 kpb. As discussed above, the probe is preferably not a complete chromosomal region, but is derived from non-contiguous regions with it. When a chromosomal region is used, then the sub-regions within are preferably represented unequally.

The term “in situ hybridization conditions” refers to conditions that facilitate hybridization of a nucleic acid to a complementary nucleic acid in an intact chromosome. Suitable in situ hybridization conditions may include both hybridization conditions and optional wash conditions, which include temperature, concentration of denaturing reagents, salts, incubation time, etc. Such conditions are known in the art.

FISH probes can be prepared according to standard procedures. See, e.g., Bolland, D. J., King, M. R., Reik, W., Corcoran, A. E., Krueger, C. Robust 3D DNA FISH Using Directly Labeled Probes. J. Vis. Exp. (78), e50587, doi:10.3791/50587 (2013). FISH, including the preparation of FISH probes, can be accomplished as described in US20130237437A1; US20120065085A1.

When using probes prepared from BAC or YAC clones and other single copy clones, a reduction in the hybridization background can be achieved by adding interspersed repeat (LINEs, SINEs) DNA in excess, which is commonly accomplished using COT1 DNA. COT1 DNA contains highly repeated DNA sequences such as SINEs, LINEs, ALUs and satellite DNA. Alternatively, human genomic DNA can be used as an agent to block such hybridization. The preferred size range is from about 200 bp to about 1000 bases, more preferably between about 400 to about 800 bp for double stranded, nick translated nucleic acids. When used as a blocking agent, the human genomic DNA can be routinely depleted of target sequences if desired.

Probe Selection

Determination of the specific probe to be used to detect the target sequence can be accomplished routinely. Probe property can be selected based on one or more of the following factors, duplex melting temperature, hairpin stability, GC content, probe complementary to an exon, probe complementary to a gene, probe complementary to intron, probe complementary to multiple regions in the genome and a proximity score. In certain embodiments, the probes can be comprised of fragments which were selected for different properties, such as the factors mentioned above. For example, fragments can be selected based on different factors, such as GC content or hairpin stability, and then pooled to make the final nucleic acid probe. See US 2011/003935 A1 for methods of selecting probes.

When a certain chromosomal region is targeted, a set of tiled or overlapping candidate nucleic acids can be selected, such as tiled YAC or BAC clones. Such tiled or overlapping nucleic acids can be constructed to unique sequences in the desired chromosomal regions. Because of the tiling or overlapping, the regions of overlap are in greater quantity than other non-overlapping regions, and thus are represented in higher amounts than in the native chromosome, particularly when amplified using a polymerase or other amplification method.

When ISH probes are made from artificial chromosomes, such as yeast artificial chromosomes (YAC), bacterial artificial chromosomes (BAC) and phage artificial chromosomes (PAC), etc., nucleotide repeats and repetitive sequences are usually present which can produce non-specific fluorescent signal and reduce the ISH detection specificity and sensitivity. Methods to reduce hybridization are known in the art, and include adding repetitive sequences to the hybridization mixture or making ISH probes that lack such sequences.

The probes can be tested to avoid using probes hybridizing to repetitive and repeat sequences. Probes can be produced using sets of various oligonucleotides which avoid repetitive sequences present in a flanking region. Such sets can be distinctly labeled, with separate or distinct reporter molecules for each probe (or set of oligonucleotides) that is aimed at the respective flanking region. Such probes can each consist of multiple labeled oligonucleotides, each hybridizing to a distinct area in a region which lacks repetitive sequences. One probe can, for example, contain from 10 up to 500 of such oligonucleotides, preferably from 50-150, each oligonucleotide, for example, being 10-20 nucleotides long. Probes can also be as much as several hundred kb, such as 10 kb, 100 kb, 150 kb, 200 kb.

As mentioned, the probes of the present invention can be produced by any suitable or known method. For example, probes can be produced using set of oligonucleotides that amplify unique, non-repetitive regions. See, e.g., WO 2014036525 A1.

Probes designed for translocations, break points, inversions, and other chromosomal rearrangements can be produced routinely. Generally, chromosomal regions flanking a breakpoint are selected. Each flanking region is labeled differently.

Probes can also be provided to identify translocations. In such cases, a balanced pair of nucleic acid probes can be produced. The probes in said pair are comparable or balanced in that they are designed to be of for example comparable size or genomic length with the final aim of facilitating the generation of signals of comparable intensity. In addition, said probes can be comparably labeled with reporter molecules resulting in signals of comparable intensity. In addition, said probes can each be labeled with a different fluorochrome, facilitating detection on one spot of different color when they co-localize when no aberration is detected. In addition, probes can be selected to react with a chromosome, at respective complementary sites that are located at comparable distances at each side of a breakpoint or breakpoint cluster region of a chromosome. The distinct and balanced pair of nucleic acid probes provided by the invention entails probes that are for example of comparable size or genomic length, each probe of the pair for example being from about 20 kb, or 15 to 30 kb, or 20 to 40 kb, or 30 to 50 kb, or 40 to 60 kb, or 50 to 70 kb, or 60 to 80 kb, or 70 to 90 kb, or 80 to 100 kb, or 100 to 500 kb or more in length. By using such a distinct and balanced pair of probes flanking a breakpoint region and not overlapping the corresponding fusion region, false-positive diagnosis in hybridization studies is avoided.

Probes can be oligonucleotides, single-stranded (ss), and double-stranded (ds).

Labeling

The labeling may be done in any convenient way. For example, in certain cases, the probes may be labeled by chemically conjugating one or more labels to the one or more double stranded polynucleotides, e.g., using the Universal Linkage System (ULS™, KREATECH Diagnostics; van Gijlswijk et al Universal Linkage System: versatile nucleic acid labeling technique Expert Rev. Mol. Diagn. 2001 1:81-91). Alternatively, the labeling may be done using nick translation, by random priming, or any other suitable method, including methods described in Ausubel et al., (Short Protocols in Molecular Biology, 3rd ed., Wiley & Sons, 1995) or Sambrook et al., (Molecular Cloning: A Laboratory Manual, Third Edition, (2001) Cold Spring Harbor, N.Y.). In certain cases, the one or more double stranded polynucleotides are labeled at multiple sites and not labeled by end labeling. As would be apparent embodiments of the method that use other labeling methods (e.g., nick translation or random priming) will produce products that differ in sequence and representation of sequence.

FN1-FGFR1 Gene Fusion

Fibronectin (FN1, also known as FN; CIG; FNZ; MSF; ED-B; FINC; GFND; LETS; GFND2)

Fibronectin (from the Latin: fibra=fiber, nexus=connection) is a glycoprotein of the extracellular matrix that binds to membrane-spanning receptor proteins called integrins. Like the integrins, fibronectin binds to other extracellular matrix components such as collagen, fibrin, and heparan sulfate proteoglycans (e.g. syndecans). Fibronectin is involved in a variety of physiological processes including tissue repair, embryogenesis, hemostasis as well as cell motility, migration and adhesion, maintenance of cell shape, wound healing, blood coagulation, host defense, and metastasis. As an unspecific opsonin, it also aids the binding of antigens to phagocytes.

Fibronectin is a soluble glycoprotein present in dimeric form in plasma, and in a dimeric or multimeric form on the cell surface and in extracellular matrix. It is involved in osteoblast compaction through the fibronectin fibrillogenesis cell-mediated matrix assembly process, which is essential for osteoblast mineralization. Fibronectins bind cell surfaces and various compounds including collagen, fibrin, heparin, DNA, and actin. It can also occur intracellularly, e.g. in thrombocytes.

The protein is encoded by the FN1 gene. It contains three regions that are subject to alternative splicing, producing at least 20 different transcript variants. The gene is located at 2q34 at NC_000002.12 (215360454..215436167, complement) (2q35 by Ensembl; 2q34 by Entrez Gene; 2q34 by HGNC).

The description of FN1 at www.ncbi.nlm.nih.gov/gene/2335; and www.uniprot.org/uniprot/P02751#section_comments are incorporated by reference in their entirety.

Mutations in the FN1 gene can lead to glomerulopathy (GFND2) or to type X Ehlers-Danlos-Sundrome.

The genomic sequence for FN1 is NG_012196.1. There are a number of different variants of FN1, including: NM_001306129.1→NP_001293058.1, fibronectin isoform 8 preproprotein; NM_001306130.1→NP_001293059.1, fibronectin isoform 9 preproprotein; NM_001306131.1→NP_001293060.1, fibronectin isoform 10 preproprotein; NM_001306132.1→NP_001293061.1, fibronectin isoform 11 preproprotein; NM_002026.2→NP_002017.1, fibronectin isoform 3 preproprotein; NM_054034.2 NP_473375.2 fibronectin isoform 7 preproprotein; NM_212474.1→NP_997639.1 fibronectin isoform 6 preproprotein; NM_212476.1→NP_997641.1 fibronectin isoform 5 preproprotein; NM_212478.1→NP_997643.1 fibronectin isoform 4 preproprotein; NM_212482.1→NP_997647.1 fibronectin isoform 1 preproprotein.

Elevated FN1 has been observed in cancer and tumors and thus a probe in accordance with the invention can be used to detect its expression levels.

Fibroblast growth factor receptor 1 (FGFR1, also known as CEK; FLG; HH2; OGD; FLT2; KAL2; BFGFR; CD331; FGFBR; FLT-2; HBGFR; N-SAM; FGFR-1; HRTFDS; bFGF-R-1)

Fibroblast growth factor 1, sometimes also referred to as basic fibroblast growth factor receptor 1, fms-related tyrosine kinase-2/Pfeiffer syndrome, and CD331, is a receptor tyrosine kinase whose ligands are specific members of the fibroblast growth factor family. FGFR1 has been shown to be associated with Pfeiffer syndrome.

The fibroblast growth factors are a highly conserved family of growth factors of which to date 23 members have been described (FGF1 to 23). The FGF growth factor molecules act through binding to specific cell surface receptor tyrosine kinases. Four receptors are known, FGFR1, FGFR2, FGFR3, FGFR4; alternative splicing of receptors 1-3 leads to additional variants (designated “b” or “c”). FGFR1 is the prototype member of the FGF receptor family. Most FGFR ligands are locally acting growth factors. FGFR1 binds to several of these FGFs as well as to the systemically acting human FGF19, FGF21 and FGF23.

The protein is a member of the fibroblast growth factor receptor (FGFR) family. FGFR family members differ from one another in their ligand affinities and tissue distribution. A full-length representative protein comprises an extracellular region, composed of three immunoglobulin-like domains, a single hydrophobic membrane-spanning segment and a cytoplasmic tyrosine kinase domain. The extracellular portion of the protein interacts with fibroblast growth factors, leading to a cascade of downstream signals, influencing mitogenesis and differentiation. FGFR1 binds both acidic and basic fibroblast growth factors. FGFR1 is a tyrosine protein kinase which, when active, phosphorylates PLCG1, FRS2, GAB1 and SHB. Ligand binding leads to the activation of several signaling cascades. Activation of PLCG1 leads to the production of the cellular signaling molecules diacylglycerol and inositol 1,4,5-trisphosphate. Phosphorylation of FRS2 triggers recruitment of GRB2, GAB1, PIK3R1 and SOS1, and mediates activation of RAS, MAPK1/ERK2, MAPK3/ERK1 and the MAP kinase signaling pathway, as well as of the AKT1 signaling pathway.

The gene for FGFR1 is located at 8p11.23-p11.22 and includes the sequences at NC_000008.11 (38411138..38468834, complement) which are incorporated by reference in their entirety. The genomic sequence for FGFR1 is NG_007729.1, which is incorporated by reference in its entirety. The gene includes a number of different NM and XM variants, including NM_023106.2, NM_023105.2, NM_001174063.1, NM_023110.2, NM_001174064.1, and NM_001174066.1.

All nucleotide and protein sequences are incorporated by reference in their entirety, including all sequences disclosed and/or referenced at the above-mentioned ncbi.nlm.nih.gov and unipot.org sites.

Gene Fusion

Several different fusion translocations between the FN1 and FGFR1 genes have been identified. Lee et al. (2015), J. Pathol., 235: 539-545 (the fusions described in the publication are incorporated by reference). Generally, a gene breakpoint at the 3′ end of the FN1 gene is fused to the 5′ end of the FGR1 gene. In one particular fusion, the FN1 gene provides its promoter to FGR1 resulting in the overexpression of the FGFR1 domains to which it is fused. In this case, the constitutively active promoter of FN1 fused to FGFR1 leads to activation of the FGFR1 kinase domain. Id. A fusion can also comprise FN1 binding domains fused to the Ig II, Ig III, TM, and kinase domains of FGFR1. Id. The resulting fusion protein can dimerize through fibronectin self-association and/or FGF ligand binding and activate the kinase domains to stimulate the FGFR1 signaling pathways, up-regulate FGF23, and then promote the activation of FN1-FGFR1. Id. Probes in accordance with the present invention can be used to detect any desired translocation of FN1 and FGFR1.

Probes

To detect the presence of the FN1-FGFR1 fusion, probes are designed which comprise sequences from FN1 and FGFR1 which preferably reside in the fusion translocation. Preferably, FN1 nucleotide sequences are labeled with one detectable label and FGFR1 are labeled with a second and different detectable label so the two sequences can be distinguished from each other. For example, as discussed above, when ISH is utilized in tissue preparations (sections, biopsy samples. etc) containing chromosomes, each probe is labeled with a different label such when a fusion translocation is present, the two different labels will appear adjacent to each other. When the fusion translocation is absent, one label will be associated with chromosome 2 and the other label with chromosome 8, with no labels appearing adjacent to each other.

In other preferred embodiments, three- and four-color probes can be used. For example, a break can occur in the FGFR1 gene and one portion of the break can be translocated to the FN1 gene. To show the presence of the break, a probe at 8p11.23 including the FGFR1 gene can be made using two colors (such as CytoOrange and CytoGreen). One probe, for example, is labeled with orange and the other with green. In a normal cell, the sequences are adjacent to each other and therefore the orange and green are visualized next to each. However, when there is a chromosome break in this region, and one portion translocates to another chromosome, the colors break apart from each other. If the green probe comprises the FGFR1 gene, then this probe will be adjacent to the new region to which it has become fused. Similarly, the FN1 region can be labeled by two probes which flank the region known to break.

A three-color probe can comprise, e.g., 1) 8p11.23 clones flanking the 3′ portion of break (FIG. 2, Table 1); 2) 8p11.23 clones flanking the 5′ portion of break (FIG. 3, Table 2); and 3) 2q35 clones (FIG. 4, Table 3) within the chromosomal region (e.g., the 5′ region) to which the 8p11.23 probe flanking the 3′ portion of break is translocated or fused. Such a probe surprisingly enabled detection of the fusion which would not have been detected with a standard break apart probe.

Table 1 (in FIG. 2) shows a list of BAC clones to the 8p11.23 region (FGFR1) flanking the 3′ portion of the chromosomal break. Table 2 (in FIG. 3) shows a list of BAC clones to the 8p11.23 region (FGFR1) flanking the 5′ portion of the break. Table 3 (in FIG. 4) shows a list of BAC clones to the 2q35 region (FN1). The numbers represent publicly available BAC clones. The BAC clones are utilized, as discussed above, to prepare ISH probes, preferably labeled probes, e.g., for FISH. For each region, one or more clones can be selected, e.g., 1, 2, 3, 4, 5, 6, 7, 8, 9, 10, etc., probes. The probes in accordance with the present invention include all combinations which can be enumerated routinely, but for brevity are not all written down here.

Disease and Drug Treatment

The probes are particularly useful for detecting chromosomal aberrations in patients. Specifically, the probes are useful as a companion diagnostic probe to determine the subset of patients with a disease or condition who would benefit from treatment with drugs that regulate the FN1-FGFR1 fusion protein and/or the pathways associated with it, such as pathways which are activated or stimulated by FGFR1. The expression of the fusion as detected by the aforementioned probes is therefore predictive of a response to a drug specifically targeting the fusion, the associated signaling pathway, or ligands to the fusion (such as FGF23).

Both of the genes involved in the FN1-FGFR1 fusion are known to play roles in disease. FN1 has been implicated in carcinoma development, has profound effects on wound healing, and is considered a potential biomarker for radioresistance. Due to the numerous physiological activities of the growth factor ligands for FGFR1, genetic abnormalities affecting the activity of this receptor can play a role in many pathological conditions, including defects of differentiation and development, angiogenesis, arteriosclerosis, coronary heart disease, peripheral artery disease, angina pectoris, wound healing and stroke. Other conditions related to abnormal activity of FGFR1 are osteoporosis, arthrosis, neural degeneration and repair, and tumor angiogenesis, cancer progression and metastasis. Somatic mutations of FGFR1 have been observed in an array of diseases. Amplification of the FGFR1 gene is common in breast and lung cancers.

The most recently discovered ligand for FGFR1, FGF23, is secreted by osteoblasts and important regulator of phosphate and vitamin D metabolism. FGFR1 abnormalities are therefore also associated with chronic kidney disease, hypophosphatemia, rickets, osteomalacia, hyperphosphatemia, phosphaturic mesenchymal tumors and some paraneoplastic syndromes such as tumor-induced osteomalacia. See Chong, W., Molinolo, A., Chen C., Collins, M. (2011), Endocr Relat Cancer. 18(3): R53-R77. doi:10.1530/ERC-11-0006. Thus, any of these disease and/or conditions, particular ones associated with aberrant phosphate metabolism, vitamin D metabolism, and/or bone disease, are likely candidates to be tested for a defects in FGFR1, particularly FN-FGFR1, and identification of the presence of the gene fusion, indicates that a drug of choice would target FGF23 or other members of its family, FGFR1, and or its signaling pathway.

As mentioned, the FN and/or FGFR probe is useful as a diagnostic to determine and guide the clinician in the choice of drug treatment. For the FN1-FGFR1 fusion, useful drugs include drugs which regulate FGF23, which regulate tyrosine kinases and/or fibronectins, such as drugs which inhibit the kinase domain of the FGFR1 receptor, inhibit polymerization of the fusion protein, inhibit FGF binding to the receptor (such as binding to the Ig II and Ig III domains), inhibit binding to the FN1 binding domains, inhibit tyrosine kinase activity, etc. Drugs include chemical compounds, antibodies, nucleic acids, peptides, etc. Generally, any drug which antagonizes FN1-FGFR1, either upstream at ligand, downstream in a signaling pathway, or the fusion itself, can be utilized in accordance with the present invention. A useful drug is an inhibitor of FGF23, by “inhibitor of FGF23,” it is meant any compound or composition that inhibits its activity, e.g., binding to the FGF23 (e.g., an antibody having specificity thereto), or an antagonist that blocks its cognate FGFR1 receptor, preventing FHF23 from binding to it.

Examples of drugs include, e.g., Dovitinib (TKI258), Ki23057, ponatinib, and AZD4547 (Katoh & Nakagama, Med. Res. Rev. (2014), 34(2):280-300); 3-Z-[1-(4-(N-((4-methyl-piperazin-1-yl)-methylcarbonyl)-N-methyl- amino)-anilino)-1-phenylmethylene]-6-methoxycarbonyl-2-indolinone, or a polymorph, metabolite or pharmaceutically acceptable salt thereof (EP147304A1, U.S. Pat. No. 7,846,936); 4-amino-5-fluoro-3-[6-(4-methylpiperazin-1-yl)-1H-benzimidazol- 2-yl]-1H-quinolin-2-one or a tautomer thereof (WO2012125812A1); RO-4396686, CHIR-258, PD 173074, methyl-piperazin-1-yl)-methylcarbonyl)-N-methyl-amino)-anilino)-1-phenylmethylene]-6-methoxycarbonyl-2-indolinone, or BIBF-1120 (US20080312260A1); heterocyclic inhibitors (WO2003020698A2); ST-571, imatinib mesylate, pyrimidine urea derivatives disclosed in WO2006000420A1, heteroaryl aryl ureas disclosed in EP1976847B1, formula I compounds disclosed in U.S. Pat. No. 8,552,002 B2. A number of newer generation FGFR inhibitor compounds are currently in clinical trials, e.g. NVP-BGJ398, a novel selective, pan-specific FGFR inhibitor (J Bone Miner Res. 2013 Apr;28(4):899-911) currently in clinical development for cancer therapy. The latter is a preferred pan-FGFR-antagonist.

Preferably, when a drug is targeted to the fusion or a member of its signaling pathway, the drug interacts and binds to either the fusion protein (e.g., Ig II or Ig III domain or FN1 binding protein), or to the constitutively activated and/or overexpressed protein, or to a protein in its signaling pathway. Preferably, when the drug is utilized to treat a disease, the drug antagonizes, inhibits, blocks, reduces, etc., the activity of the fusion or overexpressed protein or signaling pathway, thereby ameliorating symptoms of the disease.

Kits and Methods

The present invention particularly relates to kits which comprise a drug which targets FN1-FGFR1 fusion proteins, and pathways associated with its activations, and ISH probes to detect patients who would benefit from such drug treatment. The invention therefore comprises a kit comprising 1) an FN1-FGFR1 antagonist; and 2) a detectably labeled probes comprising sequences of FN1 and FGFR1 present in a translation fusion indicative of the disease. The invention also relates to methods, e.g., detecting the presence of FN1-FGFR1 in a patient with metastatic bone disease; and administering a drug which is effective to reduce or block or inhibit or antagonize the activity of the protein encoded by FN1-FGFR1.

All nucleotide and protein sequences are incorporated by reference in their entirety, including all sequences disclosed and/or referenced at the above-mentioned ncbi.nlm.nih.gov and unipot.org sites.

### EXAMPLES

Generate fluorescence labeled DNA probes by Nick Translation. DNA was extracted from identified BAC clones. Labeling was performed in two steps: 1) nick translation introducing aminoallyl-dUTP and 2) chemical coupling of an amine-reactive dye. Specifically, DNase I was used to create single-strand breaks, then DNA polymerase I was used to elongate the 3′ ends of these “nicks”, replacing existing nucleotides with new aminoallyl-dUTP. The fluorescent labeling of the probe was completed by chemical coupling of the dye. Alexa Fluor succinimidyl ester dyes react with the amines of the amino-allyl-dUTP modified DNA, thereby forming fluorescence labeled probes. Standard Ethanol precipitation method was used to isolate the fluorescence labeled probe. The probe pellet was suspended in deionized formamide/dextran sulphate.

DNA FISH Protocol. Cell slides were pretreated in pepsin solution before undergoing fixation in formaldehyde, followed by serial ethanol dehydration. The slides were denatured in formamide/saline sodium citrate (SSC) solution, followed by ice cold dehydrating ethanol series. Probes were denatured at 80° C. followed by a pre-annealing step. Pre-annealed probes were added to the denatured slides. The slides were then cover-slipped and sealed for overnight hybridization in a humidified chamber. After hybridization, slides were washed and dehydrated. At last, the slides were counterstained with anti-fade solution and mounted with coverslip for observation.

Drug Therapy.

Patients with phosphaturic mesenchymal tumors (PMTs), such as the rare paraneoplastic syndrome tumor-induced osteomalacia (TIO), appear indistinguishable based on pathology and blood and tissue biomarkers. However, ongoing trials of a pan-FGFR antagonist in both translocation positive and translocation negative individuals with PMTs have surprisingly and unexpected shown treatment responder differences. A FN1-FGFR1 translocation positive patient has responded well to pan-FGFR antagonist, NVP-BGJ398, therapy with a decrease in tumor burden. Meanwhile a patient with PMTs and negative translocation status was unresponsive to medical therapy. Such differences indicate that translocation status surprisingly indicates responsiveness to anti-FGFR therapy.

See Lee, J. C., et al., Identification of a novel FN1-FGFR1 genetic fusion as a frequent event in phosphaturic mesenchymal tumour. J Pathol, 2015. 235(4): p. 539-45; Lee, J. C., et al., Characterization of FN1-FGFR1 and novel FN1-FGF1 fusion genes in a large series of phosphaturic mesenchymal tumors. Mod Pathol, 2016. 29(11): p. 1335-1346; Collins, M. T., et al. Striking Response of Tumor-Induced Osteomalacia to the FGFR Inhibitor NVP-BGJ398 in American Society of Bone and Mineral Research Annual Meeting 2015. Seattle, Wash.

All publications cited herein are incorporated by reference in their entirety for all their teachings, and more specifically for the teachings for which they have been cited.

1. A method for treating a patient having a disease associated with a FN1-FGFR1 gene fusion, comprising
a) detecting translocation of a portion of the FGFR1 gene to the FN1 gene using chromosomal FISH probes, and
b) treating a patient with said translocation and gene fusion with at least one of: 1) a drug which inhibits FGF, 2) a drug which inhibits FGFR1, and 3) a drug which targets a signaling pathway associated with FGFR1.

2. The method of claim 1, where the drug is an inhibitor of tyrosine kinase.

3. The method of clam 1, where the drug comprises at least one of dovitinib, Ki23057, ponatinib, methyl-piperazin-1-yl)-methylcarbonyl)-N-methyl-amino)-anilino)-1-phenylmethylene]-6-methoxycarbonyl-2-indolinone, methyl-piperazin-1-yl)-methylcarbonyl)-N-methyl-amino)-anilino)-1-phenylmethylene]-6-methoxycarbonyl-2-indolinone, NVP-BGJ398, ST-571, and imatinib mesylate.

4. The method of claim 1, where the drug is an inhibitor of FGF23.

5. The method of claim 1 where the drug inhibits the activity of the protein encoded by the fusion between FN1 and FGFR1.

6. The method of claim 1, where the probes comprise 1) a probe to chromosomal region comprising FGFR1 and 2) chromosomal region comprising FN1.

7. The method of claim 1, where each probe comprises at least two different nucleic acid molecules from the same chromosomal region, where said at least two different nucleic acid molecules are represented in different quantities from each other and in different quantities then as they occur in nature, and where the chromosomal regions are 8p11.23 and 2q35.

8. The method of claim 1, where at least one nucleic acid molecule is from the 8p11.23 region and at least one nucleic acid if from the 2q35 region.

9. The method of claim 8, where each probe comprises at least 10 different nucleic acid molecules, and where at least three of said molecules are present in different quantities from each other.

10. The method of claim 1, where the probe comprises detectably-labeled nucleic molecule from the 5′ 2q35 FN1 region, the 3′ 8p11.23 FGR1 region, and the 5′ 8p11.23 region.

11. A detectably labeled DNA FISH probe comprising detectably-labeled nucleic acid molecules from the 8p11.23 chromosomal region and from the 2q35 chromosomal region, wherein the probe a FISH probe capable of detecting a fusion between the FGFR1 and FN1 genes.

12. The DNA FISH probe of claim 11, where the probe comprises at least 10 different nucleic acid molecules, and where at least three of said molecules are present in different quantities from each other.

13. The DNA FISH probe of claim 12, where at least one nucleic acid molecule is from the 8p11.23 region and at least one nucleic acid is from the 2q35 region.

14. The DNA FISH probe of claim 11, where the probe comprises detectably-labeled nucleic molecule from the 5′ 2q35 FN1 region, the 3′ 8p11.23 FGR1 region, and the 5′ 8p11.23 region.

15. The DNA FISH probe of claim 12, where the probe comprises detectably-labeled nucleic molecule from the 5′ 2q35 FN1 region, the 3′ 8p11.23 FGR1 region, and the 5′ 8p11.23 region.

16. The DNA FISH probe of claim 11, where the nucleic molecules are BAC clones.

17. The DNA FISH probe of claim 14, where the nucleic molecules are BAC clones.

18. The DNA FISH probe of claim 11, where the nucleic molecules are clones selected from Tables 1, 2, and 3.

19. The DNA FISH probe of claim 15, where the nucleic molecules are clones selected from Tables 1, 2, and 3.
